(19) United States
(12) Patent Application Publication
(10) Pub. No.: US 2025/0297322 A1
(43) Pub. Date: Sep. 25, 2025

(54) BIOMARKERS FOR MEASURING ANTICANCER DRUG RESISTANCE, AND METHOD FOR PROVIDING ANTICANCER DRUG RESISTANCE PREDICTION INFORMATION BY USING SAME

(71) Applicant: AJOU UNIVERSITY INDUSTRY-ACADEMIC COOPERATION FOUNDATION, Suwon-si, Gyeonggi-do (KR)

(72) Inventors: Hoon HUR, Seongnam-si (KR); In Hye HAM, Suwon-si (KR); Tae Hoon KIM, Suwon-si (KR)

(73) Assignee: AJOU UNIVERSITY INDUSTRY-ACADEMIC COOPERATION FOUNDATION, Suwon-si, Gyeonggi-do (KR)

(21) Appl. No.: 18/855,139

(22) PCT Filed: Apr. 27, 2023
(86) PCT No.: PCT/KR2023/005727

§ 371 Date: Oct. 8, 2024

(30) Foreign Application Priority Data
Sep. 14, 2022 (KR) 10-2022-0115906

(51) Int. Cl. C12Q 1/6886

(52) U.S. Cl. CPC C12Q 1/6886; C12Q 2600/106; C12Q 2600/158

(57) ABSTRACT

The present invention relates to biomarkers for measuring anticancer drug resistance, including genes expressed in cancer cell groups, wherein the genes cause anticancer drugs, having permeated into the cancer cell groups, to be discharged out of the cancer cell groups, and the genes are selected from the group consisting of ABCG1, p-STAT3, STAT3, p-AKT, AKT, p-ERK, ERK, β-actin, ACTA2, GAS6, FAP, PDPN, COL1A1, AXL, and combinations thereof.

### TECHNICAL FIELD

The present invention relates to biomarkers for measuring anticancer drug resistance and a method for providing anticancer drug resistance prediction information by using the same.

### BACKGROUND ART

Patients with stage 4 gastric cancer whose tumors have already progressed and cannot be resected undergo chemotherapy, such as 5-Fluorouracil (5-FU) and Cisplatin, but there is a problem that the effect is reduced due to drug resistance. As causes of the reduced drug effect, the existence and roles of not only cancer cells, but also immune cells, vascular cells, and cancer-related fibroblasts constituted around the cancer cells have been recently found, but among these cells, there are reports that cytokines or chemokines secreted from cancer-related fibroblasts are associated with increased drug resistance and poor prognosis in gastric cancer.

That is, cytokines or chemokines may function as biomarkers capable of measuring drug resistance. In general, the biomarker is an indicator capable of detecting changes in the body using proteins, DNA, RNA, metabolites, etc. For example, through genes expressed in cancer cell groups, it is possible to determine whether the cancer cell groups are disappearing, proliferating, or interacting with anticancer drugs, and the genes expressed at this time and the like may be referred to as biomarkers.

Meanwhile, gastric cancer has the fifth highest incidence rate and fourth highest mortality rate among the most common cancers worldwide, but it is known that chemotherapy and targeted therapy techniques do not significantly improve the survival probability of patients with advanced gastric cancer. This is because treatment has been focused only on cancer cells, and the influence of cancer-associated fibroblasts on cancer treatment has not been recognized. Recently, research has been conducted to analyze the influence of cancer-associated fibroblasts on cancer treatment.

As the background art of the present invention, Korean Patent Publication No. 10-0839585 relates to a method for detecting anticancer drug resistance genes using multiplex PCR and a detection kit using the same.

### DISCLOSURE

### Technical Problem

The present invention is intended to solve the problems of the above-mentioned prior art, and an object of the present invention is to provide a biomarker for measuring anticancer drug resistance and a kit for measuring anticancer drug resistance including the same.

Another object of the present invention is to provide a composition for preventing anticancer drug resistance using the biomarker for measuring anticancer drug resistance.

Yet another object of the present invention is to provide a method for providing information for predicting resistance to anticancer drug treatment using the biomarker for measuring anticancer drug resistance.

However, objects of the present invention to be achieved are not limited to the above-mentioned objects, and other objects can be present.

### Technical Solution

As a technical solution to achieve the above-mentioned technical problem, a first aspect of the present invention provides biomarkers for measuring anticancer drug resistance, including genes expressed in cancer cell groups, in which the genes cause anticancer drugs, having permeated into the cancer cell groups, to be discharged out of the cancer cell groups, and the genes are selected from the group consisting of ABCG1, p-STAT3, STAT3, p-AKT, AKT, p-ERK, ERK, β-actin, ACTA2, GAS6, FAP, PDPN, COLIAI, AXL, and combinations thereof.

According to an embodiment of the present invention, the gene may include ABCG1, but is not limited thereto.

According to an embodiment of the present invention, the cancer cell groups may include cancer cells and cancer-associated fibroblasts (CAFs) formed around the cancer cells, but are not limited thereto.

According to an embodiment of the present invention, the expression levels of the genes may be proportional to the degree of culture of the cancer cell groups, but is not limited thereto.

A second aspect of the present invention provides a kit for measuring anticancer drug resistance including the biomarker for measuring anticancer drug resistance according to the first aspect.

A third aspect of the present invention provides a composition for preventing anticancer drug resistance including an STAT3 inhibitor, in which the expression of genes expressed in cancer cell groups is inhibited by the STAT3 inhibitor.

According to an embodiment of the present invention, the composition for preventing the anticancer drug resistance may further include a gene inhibitor selected from the group consisting of an AXL inhibitor, an EKR 1 inhibitor, an EKR2 inhibitor, an AKT inhibitor, an NF-kB inhibitor, and combinations thereof, but is not limited thereto.

According to an embodiment of the present invention, the genes may include genes selected from the group consisting of ABCG1, p-STAT3, STAT3, p-AKT, AKT, p-ERK, ERK, β-actin, ACTA2, GAS6, FAP, PDPN, COLIAI, AXL, and combinations thereof, but are not limited thereto.

According to an embodiment of the present invention, the gene may include ABCG1, but is not limited thereto.

According to an embodiment of the present invention, the cancer cell groups may include cancer cells and cancer-associated fibroblasts (CAFs) formed around the cancer cells, but are not limited thereto.

A fourth aspect of the present invention provides a method for providing information for predicting resistance to treatment of anticancer drugs including measuring genes expressed in cancer cell groups: measuring anticancer drug resistance of the cancer cell groups; and analyzing a correlation between the expression levels of the measured genes and the measured anticancer drug resistance.

According to an embodiment of the present invention, the method may further include administering a gene inhibitor to the cancer cell groups: measuring genes expressed in the cancer cell groups to which the gene inhibitor has been administered; measuring anticancer drug resistance of the cancer cell groups administered with the gene inhibitor; and analyzing a correlation between the expression levels of the measured genes and the measured anticancer drug resistance, but is not limited thereto.

According to an embodiment of the present invention, the correlation between the measured expression levels of the genes and the measured anticancer drug resistance may have a positive correlation, but is not limited thereto.

The above-mentioned technical solutions are merely exemplary and should not be construed as limiting the present invention. In addition to the above-described embodiments, additional embodiments may exist in the drawings and detailed description of the invention.

### Advantageous Effects

During a conventional hepatocellular carcinoma (HCC) treating process, it was confirmed that the expression of ABCG1 was increased when treated with an Src tyrosine kinase inhibitor (TKI) and a chemotherapy Oxaliplatin, which was confirmed by RNA-sequencing to activate a Wnt signaling pathway. At this time, when the expression of ABCG1 was genetically knocked down, the tumor size was significantly reduced in a mouse in vivo model, but it was not known how the Wnt signaling pathway increased the expression of ABCG1.

In addition, resistance to a chemotherapy Cisplatin has been confirmed even in lung adenocarcinoma, which was confirmed because HOXB13 was directly bound to an ABCG1 promoter to be involved in cancer metastasis and drug resistance. However, studies have not been conducted to determine the cause of an increase in HOXB13 upon Cisplatin treatment, a signaling pathway, and what happened in a mouse in vivo model by controlling the expression of ABCG1.

In addition, resistance to 5-Fluorouracil and Oxaliplatin has been identified in colorectal cancer (CRC), which was known that ABCG1, one of ATP transporters, increased the role of discharging the drug out of a cell through a Hedgehog-GLI signaling pathway, and that a GLI transcription factor transcriptionally regulated ABC transporters. However, studies have not been conducted to determine what happened in a mouse in vivo model by controlling the expression of ABCG1.

However, researchers of the present invention confirmed through proteomic analysis that ABCG1 was increased in cancer-related fibroblasts and co-cultured cancer cells, which confirmed a novel resistance mechanism causing anticancer drug resistance, which was previously unknown. Through this, it is possible to present a method for inhibiting resistance to anticancer drugs using biomarkers for measuring anticancer drug resistance according to the present invention.

In addition, by measuring the expression of the biomarkers for measuring anticancer drug resistance, it is possible to predict resistance to anticancer drug treatment, thereby regulating anticancer drugs to be administered to patients.

However, effects obtainable herein are not limited to the effects described above, and other effects may be present.

### DESCRIPTION OF DRAWINGS

FIG. 1 is a schematic diagram illustrating a mechanism of a gene according to an embodiment of the present invention.

FIG. 2 is a diagram illustrating immunochemical staining results of cancer cell groups according to an embodiment of the present invention.

FIG. 3 is a diagram illustrating immunochemical staining results of cancer cell groups according to an embodiment of the present invention.

FIG. 4 is a diagram illustrating results of expression inhibition levels of genes according to an embodiment of the present invention.

FIGS. 5A and 5B are diagrams illustrating cell viability of cancer cell groups according to an embodiment of the present invention.

FIGS. 6A and 6B are diagrams illustrating RT-PCR of cancer cell groups according to an embodiment of the present invention.

FIGS. 7A and 7B are diagrams illustrating cell viability of cancer cell groups according to an embodiment of the present invention.

FIGS. 8A and 8B are diagrams comparing the expression between cancer-related fibroblast markers and genes according to an embodiment of the present invention.

FIGS. 9A and 9B are diagrams comparing the expression between cancer-related fibroblast markers and genes according to an embodiment of the present invention.

FIG. 10 is an image showing the expression of genes in cancer cell groups according to an embodiment of the present invention.

### BEST MODE

Hereinafter, embodiments of the present invention will be described in detail so as to be easily implemented by those skilled in the art, with reference to the accompanying drawings.

However, the present invention may be embodied in many different forms and are not limited to the embodiments to be described herein. In addition, parts not related with the description have been omitted in order to clearly describe the present invention in the drawings and throughout the present specification, like reference numerals designate like elements.

Further, throughout the present specification, when a certain part is “connected” with the other part, it is meant that the certain part may be “directly connected” with the other part and “electrically connected” with the other part with another element interposed therebetween.

Throughout this specification, it will be understood that when a member is referred to as being “on”, “above”, “under”, and “below” another member, it can be directly on the other member or intervening members may also be present.

Throughout the present specification, when a certain part “comprises” a certain component, unless otherwise disclosed to the contrary, it is meant that the part may further comprise another component without excluding another component.

The terms “about”, “substantially”, and the like to be used in the present specification are used as a numerical value or a value close to the numerical value when inherent manufacturing and material tolerances are presented in the stated meaning, and used to prevent an unscrupulous infringer from unfairly using disclosed contents in which precise or absolute numerical values are mentioned to help in the understanding of the present invention. Throughout the present specification, the term “step to” or “step of” does not mean “step for”.

Throughout the present specification, the term “combinations thereof” included in the expression of the Markush form means one or more mixtures or combinations selected from the group consisting of components described in the expression of the Markush form, and means to include at least one selected from the group consisting of the components.

Throughout the present specification, “A and/or B” means “A or B, or A and B”.

Hereinafter, biomarkers for measuring anticancer drug resistance of the present invention and a method for providing anticancer drug resistance prediction information using the same will be specifically described with reference to embodiments, Examples, and drawings. However, the present invention is not limited to these embodiments, Examples and drawings.

As a technical solution to achieve the above-mentioned technical problem, a first aspect of the present invention relates to biomarkers for measuring anticancer drug resistance, including genes expressed in cancer cell groups, in which the genes cause anticancer drugs, having permeated into the cancer cell groups, to be discharged out of the cancer cell groups, and the genes are selected from the group consisting of ABCG1, p-STAT3, STAT3, p-AKT, AKT, p-ERK, ERK, β-actin, ACTA2, GAS6, FAP, PDPN, COLIAI, AXL, and combinations thereof.

The biomarker according to the present invention means an indicator capable of detecting changes in the body using proteins, DNA, RNA, metabolites, etc. That is, through the genes expressed in the cancer cell groups, it is possible to determine whether the cancer cell groups are disappearing, proliferating, or interacting with anticancer drugs, and the genes expressed at this time may be referred to as biomarkers.

According to an embodiment of the present invention, the gene may include ABCG1, but is not limited thereto.

According to an embodiment of the present invention, the cancer cell groups may include cancer cells and cancer-associated fibroblasts (CAFs) formed around the cancer cells, but are not limited thereto.

According to an embodiment of the present invention, the expression levels of the genes may be proportional to the degree of culture of the cancer cell group, but is not limited thereto.

The ABCG1 according to the present invention was known to be expressed in cancer cells. However, the cancer-associated fibroblasts taken from the cancer cell groups are cultured and then the expression of ABCG1 is measured, and as a result, it can be confirmed that ABCG1 is expressed even in the cancer-associated fibroblasts.

According to an embodiment of the present invention, the genes may cause anticancer drugs that have permeated into the cancer cell groups to be discharged out of the cancer cell groups, but are not limited thereto.

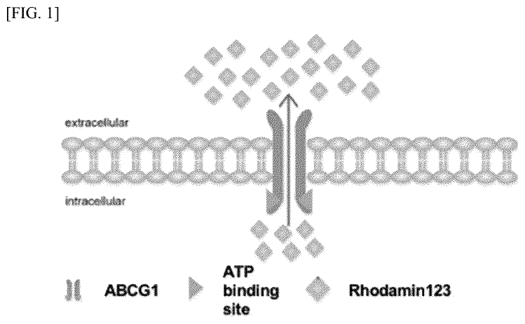

FIG. 1 is a schematic diagram illustrating a mechanism of a gene according to an embodiment of the present invention.

In general, a chemotherapy used to remove the cancer cell groups may remove the cancer cell groups by directly acting on DNA after the anticancer drugs permeate into the cancer cell groups to block the replication, transcription, and translation processes of DNA, or interfere with the synthesis of nucleic acid precursors in a metabolic pathway, and inhibit cell division. However, in the case of some cancer patients, it was confirmed that the removal degree of the cancer cell groups was low even after administration of a fixed amount of anticancer drugs. As an examined result thereof, the reason was that the genes caused the anticancer drugs having permeated into the cancer cell groups to be discharged out of the cancer cell groups.

Referring to FIG. 1, an anticancer drug, Rhodamin 123 located inside the cancer cell groups may be discharged out of the cancer cell groups by a pathway formed by ABCG1.

Although described below, the increased expression of the gene ABCG1 may be inhibited through transcription factor inhibitors AXL and STAT3, which correspond to an upper regulatory pathway.

In addition, a second aspect of the present invention relates to a kit for measuring anticancer drug resistance including the biomarkers for measuring anticancer drug resistance according to the first aspect.

As described above, the biomarkers for measuring anticancer drug resistance include genes that cause anticancer drugs having permeated into the cancer cell groups to be discharged out of the cancer cell groups. That is, it is possible to measure the resistance degree of the anticancer drugs by measuring the expression levels of the genes.

A third aspect of the present invention relates to a composition for preventing anticancer drug resistance including an STAT3 inhibitor, in which the expression of genes expressed in cancer cell groups is inhibited by the STAT3 inhibitor.

In this regard, the composition for preventing the anticancer drug resistance is intended to inhibit the expression of the biomarkers for measuring the anticancer drug resistance according to the first aspect of the present invention. When the expression of the biomarkers is inhibited, the expression of the genes is inhibited in the cancer cell groups, thereby reducing the resistance of the cancer cell groups to the anticancer drugs.

According to an embodiment of the present invention, the composition for preventing the anticancer drug resistance may further include a gene inhibitor selected from the group consisting of an AXL inhibitor, an EKR 1 inhibitor, an EKR2 inhibitor, an AKT inhibitor, an NF-kB inhibitor, and combinations thereof, but is not limited thereto.

According to an embodiment of the present invention, the genes may include genes selected from the group consisting of ABCG1, p-STAT3, STAT3, p-AKT, AKT, p-ERK, ERK, β-actin, ACTA2, GAS6, FAP, PDPN, COLIAI, AXL, and combinations thereof, but are not limited thereto.

According to an embodiment of the present invention, the gene may include ABCG1, but is not limited thereto.

The expression of the genes may be inhibited through the transcription factor inhibitors AXL and STAT3, which correspond to an upper regulatory pathway. Specifically, the expression of the genes may be regulated by inhibiting the activation of AXL and STAT3, which control the expression level of ABCG1.

According to an embodiment of the present invention, the cancer cell groups may include cancer cells and cancer-associated fibroblasts (CAFs) formed around the cancer cells, but are not limited thereto.

According to the present invention, the cancer-associated fibroblasts are fibroblasts that promote tumor initiation, growth and progression through signals that promote proliferation, angiogenesis and inflammation of cancer cells, and it was confirmed that genes that reduce the performance of anticancer drugs are expressed not only in cancer cells but also in cancer-associated fibroblasts.

In this regard, it was confirmed that the genes, specifically the genes that cause the anticancer drugs inside the cancer cell groups to be discharged to the outside, are expressed not only in cancer cells but also in cancer-related fibroblasts.

A fourth aspect of the present invention relates to a method for providing information for predicting resistance to treatment of anticancer drugs including measuring genes expressed in cancer cell groups: measuring anticancer drug resistance of the cancer cell groups; and analyzing a correlation between the expression levels of the measured genes and the measured anticancer drug resistance.

In this regard, the genes expressed in the cancer cell groups refer to the biomarkers according to the first aspect of the present invention. At this time, the biomarkers include genes that cause anticancer drugs inside the cancer cell groups to be discharged to the outside, and the higher the expression levels of the genes, the more the anticancer drugs are discharged to the outside, which means that the cancer cell groups have high anticancer drug resistance.

According to an embodiment of the present invention, the method may further include administering a gene inhibitor to the cancer cell groups: measuring genes expressed in the cancer cell groups to which the gene inhibitor has been administered; measuring anticancer drug resistance of the cancer cell groups administered with the gene inhibitor; and analyzing a correlation between the expression levels of the measured genes and the measured anticancer drug resistance, but is not limited thereto.

The expression levels of the genes expressed in the cancer cell groups and values obtained by measuring the anticancer drug resistance of the cancer cell groups may have a positive correlation. At this time, as a result of comparing the expression levels of the genes expressed in the cancer cell groups and the values obtained by measuring the anticancer drug resistance of the cancer cell groups with values of the cancer cell groups to which the gene inhibitor is not administered after administering the gene inhibitor to the cancer cell groups, it was confirmed that in a group administered with the gene inhibitor, the gene expression levels were decreased, and as a result, anticancer drug resistance was also decreased.

The gene inhibitor is intended to inhibit the expression of the genes of the biomarkers, and preferably includes an STAT3 inhibitor, and may further include an AXL inhibitor, but is not limited thereto.

According to an embodiment of the present invention, the correlation between the measured expression levels of the genes and the measured anticancer drug resistance may have a positive correlation, but is not limited thereto.

Specifically, when analyzed with published data of GSE62254 and GSE84437, it was confirmed that the expression of ABCG1 had a positive correlation with cancer-associated fibroblast markers in gastric cancer patients, and at this time, when the expression level of ABCG1 and the expression of cancer-associated fibroblasts were high, the prognosis of gastric cancer was poor. However, the expression level of ABCG1 may be controlled by transcription factor inhibitors AXL and STAT3, which correspond to an upper regulatory pathway that regulates the action of ABCG1.

The above-mentioned technical solutions are merely exemplary and should not be construed as limiting the present invention. In addition to the above-described embodiments, additional embodiments may exist in the drawings and detailed description of the invention.

Hereinafter, the present invention will be described in more detail with reference to the following Examples, but the following Examples are only for illustrative purposes and are not intended to limit the scope of the present invention.

### EXAMPLE

The expression of genes such as ABCG1 was investigated using cancer cells SNU668, MKN1, GSE62254, and GSE84437.

### Experimental Example 1

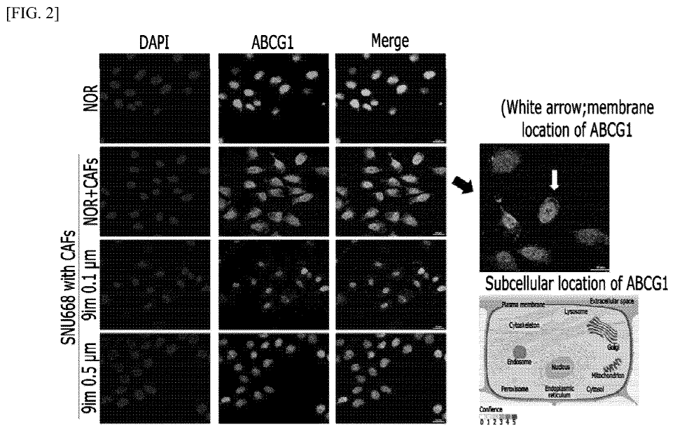

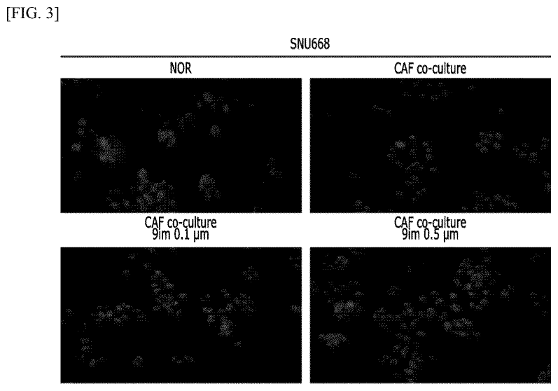

FIGS. 2 and 3 are diagrams illustrating immunochemical staining results of cancer cell groups according to an embodiment of the present invention. In this regard, the arrow in FIG. 2 indicated the expression of ABCG1, and since the ABCG1 was mainly expressed in the cell membrane, the arrow indicated a portion where the expression of ABCG1 was prominent.

Referring to FIGS. 2 and 3, it can be confirmed that the color development points of DAPI and ABCG1 are consistent with each other.

### Experimental Example 2

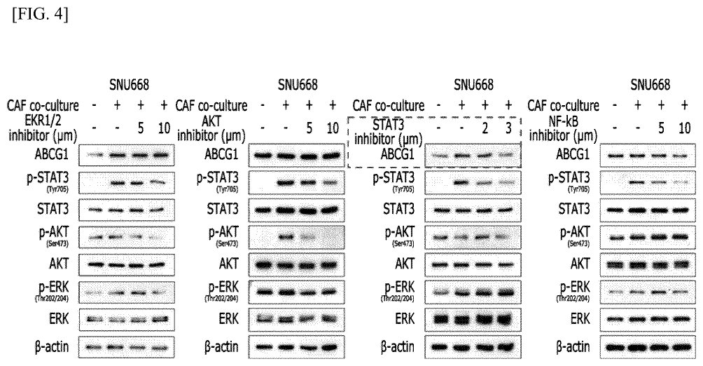

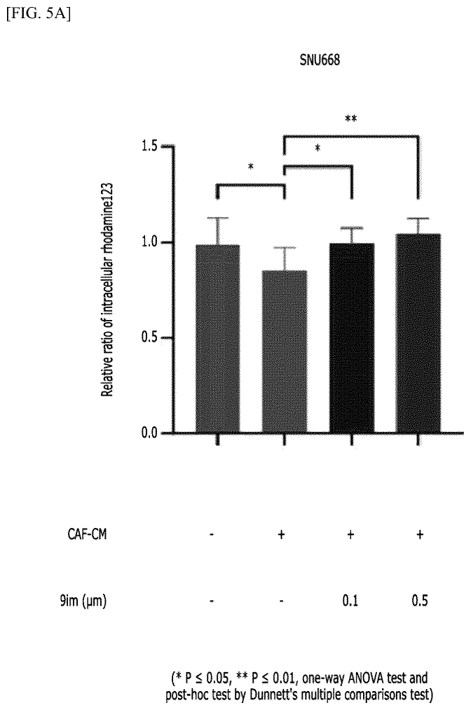

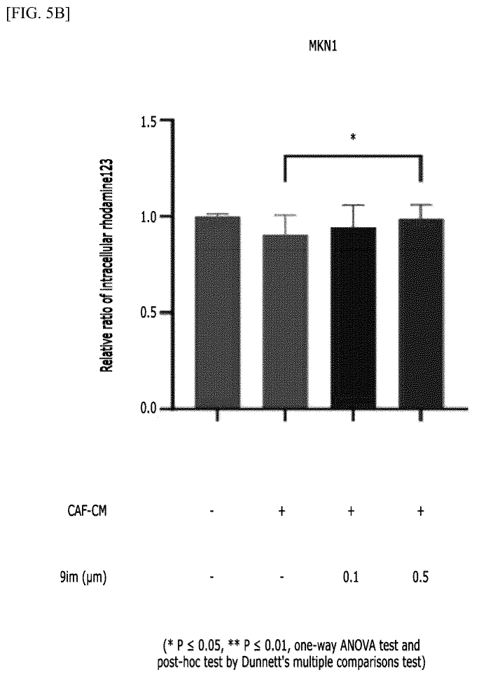

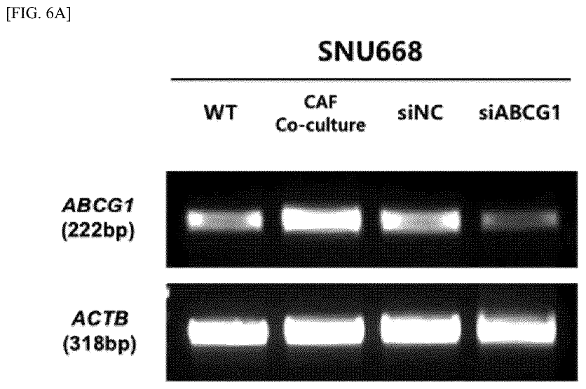

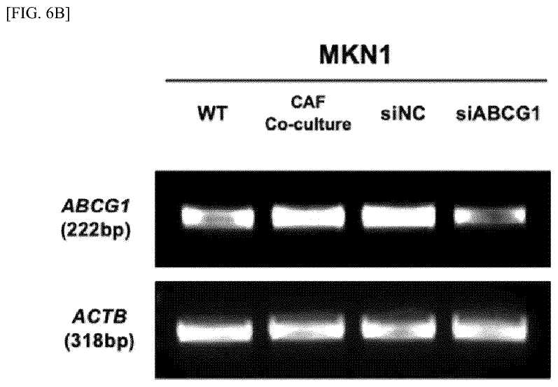

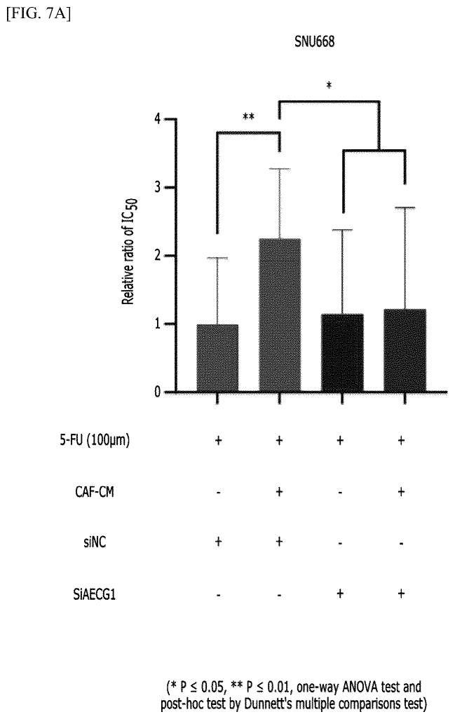

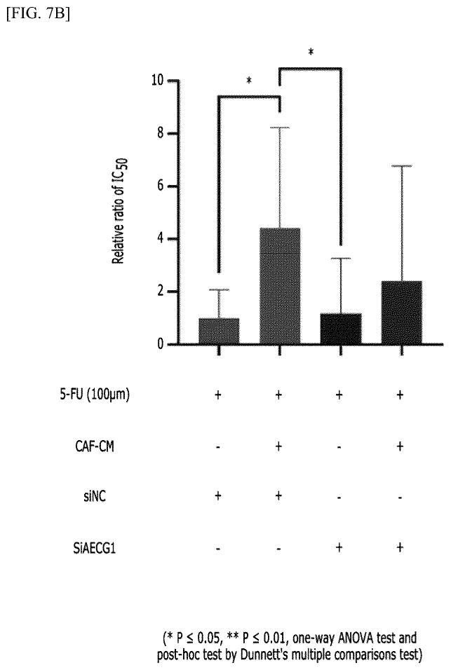

FIG. 4 illustrates results of gene expression inhibition levels of genes according to an embodiment of the present invention, FIGS. 5A, 5B, 7A and 7B illustrate cell viability of cancer cell groups according to an embodiment of the present invention, and FIGS. 6A and 6B illustrate RT-PCR of cancer cell groups according to an embodiment of the present invention.

Referring to FIGS. 4 to 7B, it can be confirmed that the expression of ABCG1 is inhibited by STAT3.

Specifically, referring to FIG. 4, when a gastric cancer cell line SNU668 is co-cultured with CAF, it causes activation of downstream signaling pathways known for AXL occurred in the gastric cancer cell line, which is a result of treating the gastric cancer cell line with inhibitors corresponding to ERK, AKT, STAT3, and NF-kB, respectively. As a result, it means that when the gastric cancer cell line is treated with the STAT3 inhibitor, the expression of ABCG1 may be inhibited in a concentration-dependent manner.

In addition, referring to FIG. 5B, when the gastric cancer cell line is co-cultured with CAF, an increase in protein and gene levels of ABCG1 may be confirmed, and it can be confirmed that ABCG1 has an anticancer drug discharge function even in a normal state when not co-cultured with the CAF.

In addition, referring to FIG. 7B, when the gastric cancer cell line is treated with CAF-CM (CAF conditioned medium) under a condition of treating an anticancer drug 5-FU, anticancer drug resistance occurs and thus the survival of cancer cells is increased, but it can be seen that when the ABCG1 gene is knocked down in the gastric cancer cell line, anticancer drug resistance may be inhibited.

### Experimental Example 3

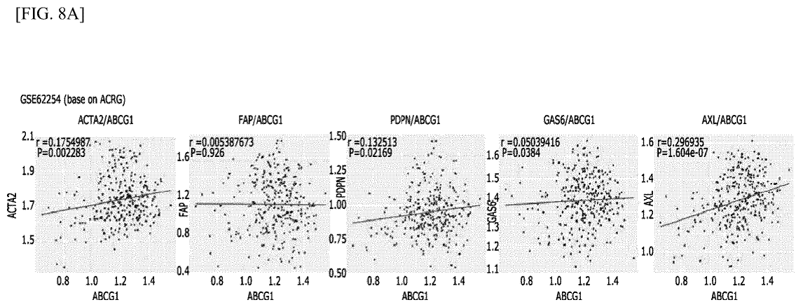

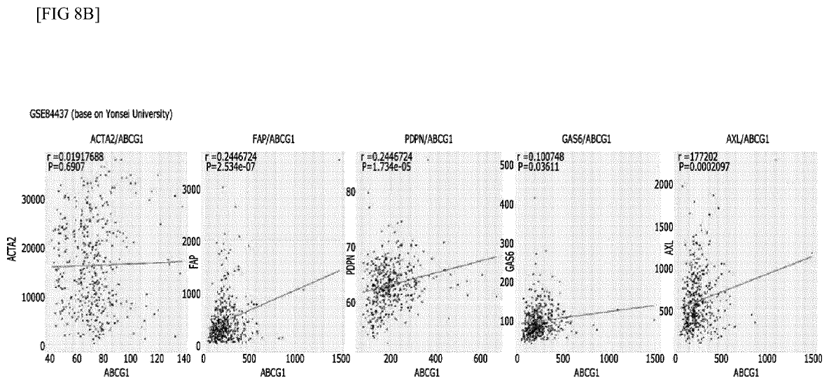

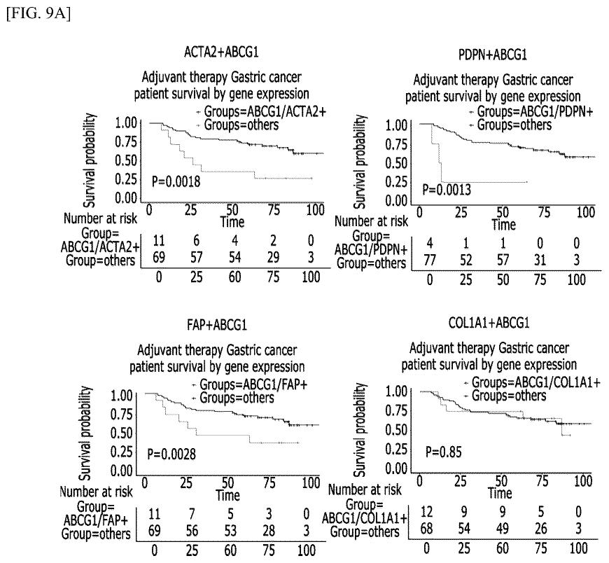

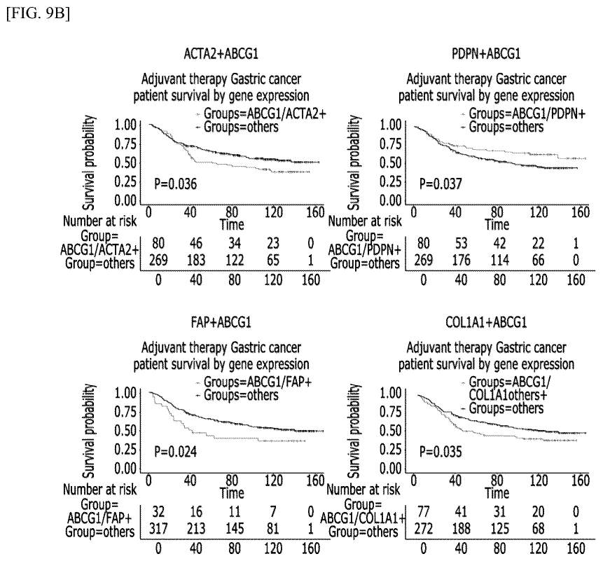

FIGS. 8A, 8B, 9A, and 9B compare the expression between cancer-related fibroblast markers and genes according to an embodiment of the present invention. At this time, in FIGS. 9A and 9B, lower tables at the graphs indicate an ABCG1/ACTA2+ group (upper left), an ABCG1/PDPN+ group (upper right), an ABCG1/FAP+ group (lower left), an ABCG1/COL1A1 group (lower right), and other groups (blue graphs for each graph).

Referring to FIGS. 8A to 9B, it can be confirmed that the expression of ABCG1 in GSE62254 and GSE64437 commonly has a positive correlation with the expression of PDPN and AXL.

In this regard, PDPN is a gene marker of CAF, and inhibiting GAS6 protein capable of activating AXL in the gastric cancer cell line in CAF may regulate ABCG1 expression in the gastric cancer cell line rather than inhibiting PDPN in CAF.

### Experimental Example 4

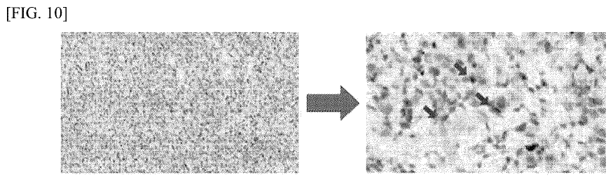

FIG. 10 is an image showing the expression of genes in cancer cell groups according to an embodiment of the present invention. In this regard, FIG. 10 shows immunohistochemical staining of gastric cancer tissue of a patient who undergoes gastric cancer resection surgery, and a red arrow in FIG. 10 represents the expression of ABCG1 in a cancer cell line.

Referring to FIG. 10, the expression of ABCG1 may be confirmed even when gastric cancer cells are resected, which means that ABCG1 is expressed not only in gastric cancer cells but also in gastric cancer CAFs around the gastric cancer cells.

The aforementioned description of the present invention is to be exemplified, and it will be understood by those skilled in the art that the present invention can be easily modified in other detailed forms without changing the technical spirit or required features of the present invention. Therefore, it should be appreciated that the embodiments described above are illustrative in all aspects and are not restricted. For example, each component described as a single form may be implemented in a distributed manner, and likewise, components described as distributed may be implemented in a combined manner.

The scope of the present invention is represented by appended claims to be described below rather than the detailed description, and it is to be interpreted that the meaning and scope of the claims and all the changes or modified forms derived from the equivalents thereof come within the scope of the present invention.

1. A biomarker for measuring anticancer drug resistance comprising genes expressed in cancer cell groups,
wherein the genes cause anticancer drugs, having permeated into the cancer cell groups, to be discharged out of the cancer cell groups, and
in the biomarker for measuring anticancer drug resistance,
the genes are selected from the group consisting of ABCG1, p-STAT3, STAT3, p-AKT, AKT, p-ERK, ERK, β-actin, ACTA2, GAS6, FAP, PDPN, COLIAI, AXL, and combinations thereof.

2. The biomarker for measuring anticancer drug resistance of claim 1, wherein the gene includes ABCG1.

3. The biomarker for measuring anticancer drug resistance of claim 1, wherein the cancer cell groups include cancer cells and cancer-associated fibroblasts (CAFs) formed around the cancer cells.

4. The biomarker for measuring anticancer drug resistance of claim 1, wherein expression levels of the genes are proportional to a degree of culture of the cancer cell groups.

5. A kit for measuring anticancer drug resistance comprising the biomarker for measuring anticancer drug resistance according to claim 1.

6. A composition for preventing anticancer drug resistance comprising an STAT3 inhibitor,
wherein an expression of genes expressed in cancer cell groups is inhibited by the STAT3 inhibitor.

7. The composition for preventing anticancer drug resistance of claim 6, further comprising:
a gene inhibitor selected from the group consisting of an AXL inhibitor, an EKR 1 inhibitor, an EKR2 inhibitor, an AKT inhibitor, an NF-kB inhibitor, and combinations thereof.

8. The composition for preventing anticancer drug resistance of claim 6, wherein the genes are selected from the group consisting of ABCG1, p-STAT3, STAT3, p-AKT, AKT, p-ERK, ERK, β-actin, ACTA2, GAS6, FAP, PDPN, COLIAI, AXL, and combinations thereof.

9. The composition for preventing anticancer drug resistance of claim 8, wherein the gene includes ABCG1.

10. The composition for preventing anticancer drug resistance of claim 6, wherein the cancer cell groups include cancer cells and cancer-associated fibroblasts (CAFs) formed around the cancer cells.

11. A method for providing information for predicting resistance to treatment of anticancer drugs comprising:
measuring genes expressed in cancer cell groups;
measuring anticancer drug resistance of the cancer cell groups; and
analyzing a correlation between expression levels of the measured genes and the measured anticancer drug resistance.

12. The method for providing information for predicting resistance to treatment of anticancer drugs of claim 11, further comprising:
administering a gene inhibitor to the cancer cell groups; measuring genes expressed in the cancer cell groups to which the gene inhibitor has been administered; measuring anticancer drug resistance of the cancer cell groups administered with the gene inhibitor; and analyzing a correlation between expression levels of the measured genes and the measured anticancer drug resistance.

13. The method for providing information for predicting resistance to treatment of anticancer drugs of claim 11, wherein the correlation between the expression levels of the measured genes and the measured anticancer drug resistance has a positive correlation.
